(19) United States
(12) Patent Application Publication
(10) Pub. No.: US 2004/0161848 A1
(43) Pub. Date: Aug. 19, 2004

(54) Adenoviral vector and related system and methods of making and use

(75) Inventor: Imre Kovesdi, Rockville, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: GenVec, Inc., Gaithersburg, MD

(21) Appl. No.: 10/778,832

(22) Filed: Feb. 13, 2004

Related U.S. Application Data
(63) Continuation of application No. PCT/US02/29111, filed on Sep. 13, 2002.
(60) Provisional application No. 60/318,997, filed on Sep. 13, 2001.

(51) Int. Cl. A61K 48/00; C12N 15/861

(52) U.S. Cl. 435/456; 424/93.2

(57) ABSTRACT

An adenoviral vector comprising an adenoviral genome comprising (i) at least one deletion in a region of the adenoviral genome selected from the group consisting of E1, E2A and E4, (ii) (a) at least one deletion in the VAI gene of the adenoviral genome, alone or in further combination with at least one deletion in the VAII gene of the adenoviral genome, (b) a recombinant VAI gene, alone or in further combination with a recombinant VAII gene, wherein the recombinant gene comprises either of a regulatable promoter in place of the native promoter or a mutated native promoter and 5′ to the mutated native promoter, a pol II promoter, or (c) a dominant negative, double-stranded, RNA-dependent protein kinase (PKR) and, optionally, (iii) a polymerase II (pol II) construct comprising a pol II promoter operably linked to a coding region and/or a polymerase III (pol III) construct comprising a pol III promoter operably linked to a coding region, as well as a system comprising such an adenoviral vector and a cell line that complements the adenoviral vector, and related systems and methods.

### TECHNICAL FIELD OF THE INVENTION

This patent application is a continuation of International Patent Application No. PCT/US02/29111, filed Sep. 13, 2002, which designates the U.S., which claims the benefit of U.S. Provisional Patent Application No. 60/318,997, filed Sep. 13, 2001.

### TECHNICAL FIELD OF THE INVENTION

This invention pertains to an adenoviral vector, a system comprising such an adenoviral vector and a complementing cell line, a method of making an adenoviral vector, a method of making a complementing cell line for the adenoviral vector, and a method of expressing polymerase II (pol II) and/or polymerase III (pol III) constructs.

### BACKGROUND OF THE INVENTION

Adenoviral vectors are ideally suited to allow for short-term gene expression. However, securing long-term gene expression has been a problem with first generation (e.g., E1 deficient) and second generation (e.g., E1 and E4 deficient) vectors. The newer amplicon vectors have been shown to express genes for an extended period of time in several animal species and tissues. However, these vectors are very difficult to produce in large quantities. Furthermore, amplicon vectors are produced with helper adenoviruses, which contaminate the final product with helper virus particles and replication-competent adenovirus.

The invention seeks to avoid the above-mentioned shortcomings of adenoviral vectors. Thus, it is an object of the invention to provide an adenoviral vector that avoids at least some of the shortcomings of currently available vectors, as well as a system comprising such an adenoviral vector and a complementing cell line, a method of making such an adenoviral vector, a method of making a complementing cell line for the adenoviral vector, and a method of expressing pol II and/or pol III constructs using such an adenoviral vector. These and other objects, as well as advantages and additional inventive features of the invention, will be apparent from the description of the invention provided herein.

### SUMMARY OF THE INVENTION

The invention provides an adenoviral vector comprising an adenoviral genome comprising (i) at least one deletion in a region of the adenoviral genome selected from the group consisting of E1, E2A and E4, (ii) at least one deletion in the VAI gene of the adenoviral genome, alone or in further combination with at least one deletion in the VAII gene of the adenoviral genome, and, optionally, (iii) a pol II construct comprising a pol II promoter operably linked to a coding region and/or a pol III construct comprising a pol III promoter operably linked to a coding region. The invention further provides a system comprising such an adenoviral vector and a cell line that complements the adenoviral vector, wherein the cell line comprises the adenoviral vector. The cell line can express the VAI gene, alone or in further combination with the VAII gene as appropriate, each under the control of the gene's native promoter or under the control of a regulatable promoter.

The invention also provides an adenoviral vector comprising an adenoviral genome comprising (i) at least one deletion in a region of the adenoviral genome selected from the group consisting of E1, E2A and E4, (ii) a recombinant VAI gene, alone or in further combination with a recombinant VAII gene, wherein the recombinant gene comprises either of a regulatable promoter in place of the gene's native promoter or a mutated native promoter and, 5′ to the mutated native promoter, a pol II promoter, and, optionally, (iii) a pol II construct comprising a pol II promoter operably linked to a coding region and/or a pol III construct comprising a pol III promoter operably linked to a coding region. The invention further provides a system comprising such an adenoviral vector and a cell line, wherein the cell line comprises the adenoviral vector.

Yet another adenoviral vector provided by the invention is one that comprises an adenoviral genome comprising (i) at least one deletion in a region of the adenoviral genome selected from the group consisting of E1, E2A and E4, (ii) an oligonucleotide sequence encoding a dominant negative, double-stranded, RNA-dependent protein kinase (PKR; also referred to as DAI, p68, DsI or P1/eIF-2), and, optionally, (iii) a pol II construct comprising a pol II promoter operably linked to a coding region and/or a pol III construct comprising a pol III promoter operably linked to a coding region. The invention further provides a system comprising such an adenoviral vector and a cell line, wherein the cell line comprises the adenoviral vector.

A system comprising an adenoviral vector comprising (i) an adenoviral genome comprising at least one deletion in a region of the adenoviral genome selected from the group consisting of E1, E2A and E4, and, optionally, a pol II construct comprising a pol II promoter operably linked to a coding region and/or a pol III construct comprising a pol III promoter operably linked to a coding region, and (ii) a cell line that complements the adenoviral vector, wherein the cell line expresses a dominant negative, double-stranded PKR and comprises the adenoviral vector, is also provided by the invention.

Also provided by the invention is a method of making such an adenoviral vector. In one embodiment, the method comprises (i) introducing at least one deletion in a region of the adenoviral genome selected from the group consisting of E1, E2A and E4, (ii) introducing at least one deletion in the VAI gene of the adenoviral genome, alone or in further combination with at least one deletion in the VAII gene of the adenoviral genome, and, optionally, (iii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order. In another embodiment, the method comprises (i) introducing at least one deletion in each of the E1, E4, VAI and VAII regions of the adenoviral genome, and, optionally, (ii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order. In still yet another embodiment, the method comprises (i) introducing at least one deletion in a region of the adenoviral genome selected from the group consisting of E1, E2A and E4, (ii) either (a) introducing a mutation into the native promoter of the VAI gene and/or the VAII gene, and 5′ to the mutated native promoter, introducing a pol II promoter, or (b) substituting a regulatable promoter for the native promoter of the VAI gene and/or the VAII gene, and, optionally, (iii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order. In a further embodiment, the method comprises (i) introducing at least one deletion in the VAI or VAII encoding region of the adenoviral genome and at least one deletion in a region of the adenoviral genome selected from the group consisting of E1, E2A and E4, (ii) replacing or mutating at least a portion of the VAI promoter (pol III promoter (e.g., A box)) of the adenoviral genome and operably linking it with a regulatable promoter, such as the sheep metallothionine promoter, and, optionally, (iii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order. In a further embodiment, the method comprises (i) introducing at least one deletion in a region of the adenoviral genome selected from the group consisting of E1, E2A and E4, (ii) introducing an oligonucleotide sequence encoding a dominant negative, double-stranded PKR kinase into the adenoviral vector. The method can further comprise propagating the adenoviral vector in a complementing cell line.

A method of expressing a pol II construct comprising a pol II promoter operably linked to a coding region and/or a pol III construct comprising a pol III promoter operably linked to a coding region in a mammalian cell is also provided. The method comprises contacting the mammalian cell with an above-described adenoviral vector, which comprises a pol II construct comprising a pol II promoter operably linked to a coding region and/or a pol III construct comprising a pol III promoter operably linked to a coding region, whereupon the mammalian cell internalizes the adenoviral vector and expresses the pol II construct and/or the pol III construct in the mammalian cell.

Still also provided is a method of making a cell line that complements an above-described adenoviral vector. The method comprises (i) introducing into the cell those regions of an adenoviral genome selected from the group consisting of E1, E2A and E4 that have been disrupted in the adenoviral vector, and (ii) introducing into the cell an adenoviral VAI coding region operably linked to a promoter and/or an adenoviral VAII coding region operably linked to a promoter, whereupon the cell expresses those regions of the adenoviral genome and the adenoviral VAI coding region and/or the adenoviral VAII coding region, as have been introduced into the cell, wherein each of the VAI and/or the VAII coding region(s) is under the control of its native promoter or an inducible promoter, and desirably wherein there is no overlap with respect to at least one essential gene function between the adenoviral genome of the adenoviral vector and those regions of the adenoviral genome and the adenoviral VAI coding region, alone or in further combination with the adenoviral VAII coding region, as have been introduced into the cell for generation of replication-competent adenovirus, with the proviso that (i) and (ii) can be carried out in either order.

### DETAILED DESCRIPTION OF THE INVENTION

The invention provides adenoviral vectors that have characteristics in common with amplicons, yet desirably few or none of their shortcomings, and that offer persistent expression. The adenoviral vectors achieve persistent expression by inducing less of an immune response in vivo. Accordingly, the invention provides an adenoviral vector comprising an adenoviral genome comprising (i) at least one deletion in a region of the adenoviral genome selected from the group consisting of E1, E2A and E4 and (ii) at least one deletion in the VAI gene of the adenoviral genome, alone or in further combination with at least one deletion in the VAII gene of the adenoviral genome. Another adenoviral vector provided by the invention is one that comprises an adenoviral genome comprising (i) at least one deletion in a region of the adenoviral genome selected from the group consisting of E1, E2A and E4, and (ii) a recombinant VAI gene, alone or in further combination with a recombinant VAII gene, wherein the recombinant gene comprises either of a regulatable promoter in place of the gene's native promoter or a mutated native promoter and, 5′ to the mutated native promoter, a pol II promoter. Yet another adenoviral vector provided by the invention is one that comprises an adenoviral genome comprising (i) at least one deletion in a region of the adenoviral genome selected from the group consisting of E1, E2A and E4, and (ii) an oligonucleotide sequence encoding a dominant negative, double-stranded PKR kinase. The expression of the dominant negative, double-stranded PKR kinase can be constitutive, inducible or repressible as desired. While the adenoviral vector can consist of a recombinant adenoviral genome, desirably the adenoviral vector is encapsidated, i.e., a viral particle or virion.

The at least one deletion can be any deletion in an indicated region of the adenoviral genome, the VAI gene and/or the VAII gene. A deletion in an indicated region of the adenoviral genome can be a deletion in or affecting a gene, such as a gene required for adenoviral replication (see, e.g., International Patent Application Publication No. WO 95/34671), such that the adenoviral vector is a replication-deficient adenoviral vector. The deletion can result in a disruption of function. By “disruption of function” is meant that the indicated gene or region functions at a level substantially below normal, such as by at least about 5-fold, at least about 10-fold, or has ceased to function. Alternatively, the disruption of function can result from substitution(s), insertion(s), deletion(s), and/or inversion(s), such as in a coding sequence and/or a regulatory element of the indicated gene or indicated region of the adenoviral genome.

By “E1 region” is meant the region of the adenoviral genome from around 1.56 to around 9.74 map units (mu) or from around 560 to around 3,506 base pairs (bp). The E1 region comprises the E1A region (from around 1.56 to around 4.29 mu or from around 560 to around 1,545 bp) and the E1B region (from around 4.76 to around 9.74 mu or from around 1,714 to around 3,506 bp). By “E2A region” is meant the region of the adenoviral genome from around 62.34 to around 66.76 mu or from around 22,443 to around 24,032 bp. By “E3 region” is meant the region of the adenoviral genome from around 79.30 to around 85.66 mu or from around 28,547 to around 30,839 bp. The aforementioned limits of the indicated adenoviral regions are based on the Ad2 and Ad5 adenoviral genome maps and may vary slightly from one adenoviral serotype to another.

The VA region in the Ad2 genome is contained in the Sal I-Hin dIII fragment (from around 9,831 bp to 11,555 bp). More specifically, the VAI gene is located from around 10,610 bp to around 10,766 bp, and the VAII gene is located from around 10,866 bp to around 11,023 bp.

Any subtype, mixture of subtypes or chimeric adenovirus can be used as the source of DNA for generation of the adenoviral vector comprising an adenoviral genome comprising at least one deletion in a region of the adenoviral genome selected from the group consisting of E1, E2A and E4. The generation of at least one deletion in the E1, E2A and/or E4 region of the adenoviral genome can be done in accordance with the methods set forth in International Patent Application Publication No. WO 95/34671. In a preferred embodiment of the invention, the adenoviral vector comprises at least one deletion in each of two regions of the adenoviral genome selected from the group consisting of E1, E2A and E4. The generation of at least one deletion in the VAI gene, alone or in further combination with at least one deletion in the VAII gene, of the adenoviral genome is exemplified herein. In a preferred embodiment of the invention, the adenoviral vector comprises at least one deletion in each of the E1 and E4 regions and the VAI and VAII genes of the adenoviral genome.

An adenoviral vector described above, such as one that comprises at least one deletion in the E1 region and at least one deletion in the E4 region, may not require VA RNA and will persist without causing inflammation and/or innate immune responses against the adenoviral vector components. Such a vector could be useful in a composition for treating diseases of the eye, ear; etc., and metabolic diseases. Such a vector also could be useful for inducing a specific immune response against epitopes incorporated into the viral coat proteins and/or expressed from the vector genome, such as a vaccine composition, such as a vaccine against a virus, e.g., hepatitis A, hepatitis B, hepatitis C, Herpes simplex (HSV), human immunodeficiency virus (HIV), or malaria, or a bacterium or parasite, e.g., pseudomonas, anthrax or plague. An adenoviral vector described above, such as one that comprises at least one deletion in the E1 region alone, may cause interferon release and inflammation in the absence of VA RNA. Such a vector could be useful in a composition for inducing an immune response and/or an innate immune response, such as a vaccine composition.

Optionally, the adenoviral vector further comprises a pol II construct comprising a pol II promoter operably linked to a coding region and/or a pol III construct comprising a pol III promoter operably linked to a coding region. The operable linkage of a coding region to a promoter is within the skill in the art. Preferably, the coding region operably linked to the pol III promoter encodes an antisense RNA molecule, a ribozyme, a small interfering RNA (siRNA), or any other RNA interfering molecule. Preferably, the pol III promoter is a VA RNA promoter, a transfer RNA promoter or a small nuclear RNA promoter. Preferred pol II promoters include a constitutive promoter (e.g., the Rous sarcoma virus long terminal repeat (RSV LTR) promoter/enhancer or the cytomegalovirus major immediate early gene (CMV IE) promoter, which is particularly preferred), an inducible promoter, (e.g., a growth hormone promoter, metallothionein promoter, heat shock protein promoter, EIB promoter, hypoxia induced promoter, or MLP promoter and tripartite leader), an inducible-repressible promoter, a developmental stage-related promoter (e.g., a globin gene promoter), or a tissue specific promoter (e.g., a smooth muscle cell α-actin promoter, VEGF receptor promoter, myosin light-chain 1A promoter, or vascular endothelial cadherin promoter). In some instances, host-native promoters can be preferred over non-native promoters, particularly where strict avoidance of gene expression silencing due to host immunological reactions is desirable.

The adenoviral vector can be subject to any number of additional or alternative modifications. For example, a particularly preferred vector includes or expresses a modified adenoviral protein, non-adenoviral protein, or both, which increases the efficiency that the vector infects cells as compared to wild-type adenovirus, allows the vector to infect cells which are not normally infected by wild-type adenovirus, results in a reduced host immune response in a mammalian host as compared to wild-type adenovirus, or any combination thereof. Any suitable type of modification can be made to the vector, and several suitable modifications are known in the art. For example, the adenoviral vector coat protein can be modified. Examples of such modifications include modifying the adenoviral fiber, penton, pIX, pIIIa, or hexon proteins, and/or insertions of various native or non-native ligands into portions of such coat proteins. Manipulation of such coat proteins can broaden the range of cells infected by a viral vector or enable targeting of a viral vector to a specific cell type. One direct result of manipulation of the viral coat is that the adenovirus can bind to and enter a broader range of eukaryotic cells than a wild-type virus. Examples of adenoviruses including such modifications are described in International Patent Application WO 97/20051. Reduction of immune response against the adenovirus also or alternatively can be obtained through the methods described in U.S. Pat. No. 6,093,699. In other embodiments, the viral coat is manipulated such that the virus is “targeted” to a particular cell type, e.g., those cells expressing unique receptors. Examples of such modified adenoviral vectors are described in U.S. Pat. Nos. 5,559,099, 5,731,190, 5,712,136, 5,770,442, 5,846,782, 5,962,311, 5,965,541, and 6,057,155 and International Patent Application Publication Nos. WO 96/07734, WO 96/26281, WO 97/20051, WO 98/07865, WO 98/07877, WO 98/40509, WO 98/54346, and WO 00/15823. Other adenoviral vector protein modifications that decrease the potential for immunological recognition by the host and resultant coat-protein directed neutralizing antibody production, are described in, e.g., International Patent Application Publication Nos. WO 98/40509 and WO 00/34496. The adenoviral vector can be packaged as a virion. In other embodiments of the coat, modifications can be used to incorporate immunological epitopes into the fiber, penton, hexon or pIX proteins. Such a vector could be useful for inducing a specific immune response against the epitopes incorporated into the viral coat proteins for vaccine production. See, e.g., International Patent Application Publication No. WO 01/58478, published Aug. 16, 2001.

Thus, in view of the above, the invention also provides a method of making an above-described adenoviral vector. In one embodiment, the method comprises (i) introducing at least one deletion in a region of the adenoviral genome selected from the group consisting of E1, E2A and E4, (ii) introducing at least one deletion in the VAI gene of the adenoviral genome, alone or in further combination with at least one deletion in the VAII gene of the adenoviral genome, and, optionally, (iii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order. The method can further comprise (iv) propagating the adenoviral vector in a complementing cell line.

In another embodiment of the method of a making an above-described adenoviral vector, the method comprises (i) introducing at least one deletion in each of the E1, E4, VAI and VAII regions of the adenoviral genome, and, optionally, (ii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order. The method can further comprise (iii) propagating the adenoviral vector in a complementing cell line.

In still yet another embodiment of the method of making an above-described adenoviral vector, the method comprises (i) introducing at least one deletion in a region of the adenoviral genome selected from the group consisting of E1, E2A and E4, (ii) either (a) introducing a mutation into the native promoter of the VAI gene and/or the VAII gene, and 5′ to the mutated native promoter, introducing a pol II promoter, or (b) substituting a regulatable promoter for the native promoter of the VAI gene and/or the VAII gene, and, optionally, (iii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order. The method can further comprise (iv) propagating the adenoviral vector in a complementing cell line.

In yet another embodiment of the method of making an above-described adenoviral vector, the method comprises (i) introducing at least one deletion in the VAI or VAII encoding region of the adenoviral genome and at least one deletion in a region of the adenoviral genome selected from the group consisting of E1, E2A and E4, (ii) replacing or mutating at least a portion of the VAI promoter (pol III promoter (e.g., A box)) of the adenoviral genome and operably linking it with a regulatable promoter, such as the sheep metallothionine promoter, which can be induced (the sheep metallothionine promoter can be induced by zinc) at the beginning of an adenovirus production cycle in a complementing cell line, and, optionally, (iii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order. The method can further comprise (iv) propagating the adenoviral vector in a complementing cell line, which does not express VAI and/or VAII in the presence of a repressor of the regulatable promoter or constitutively expresses VAI and/or VAII at a reasonable level.

In still yet another method of making an above-described adenoviral vector, the method comprises (i) introducing at least one deletion in a region of the adenoviral genome selected from the group consisting of E1, E2A and E4, (ii) introducing an oligonucleotide sequence encoding a dominant negative, double-stranded PKR kinase into the adenoviral vector, and, optionally, (iii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order. The method can further comprise (iv) propagating the adenoviral vector in a complementing cell line. The expression of the dominant negative, double-stranded PKR kinase can be constitutive, inducible or repressible as desired.

A system comprising an above-described adenoviral vector and a cell line that complements the adenoviral vector, wherein the cell line comprises the adenoviral vector, is also provided by the invention. When the adenoviral vector comprises an adenoviral genome comprising (i) at least one deletion in a region of the adenoviral genome selected from the group consisting of E1, E2A and E4 and (ii) at least one deletion in the VAI gene of the adenoviral genome, alone or in further combination with at least one deletion in the VAII gene of the adenoviral genome, and, optionally, (iii) a pol II construct comprising a pol II promoter operably linked to a coding region and/or a pol III construct comprising a pol III promoter operably linked to a coding region, the cell line produces VAI, alone or in further combination with VAII as appropriate. The VAI and/or VAII gene can be under the control of the gene's native promoter, such that the encoded product is constitutively expressed. Alternatively, the VAI and/or VAII gene is under the control of a regulatable promoter, such as one that comprises an element that binds to a tetracycline repressor, such as one that binds to a tetracycline repressor interferes with the split binding sites of the transcription factor TFIIID. When the adenoviral vector comprises an adenoviral genome comprising (i) at least one deletion in a region of the adenoviral genome selected from the group consisting of E1, E2A and E4, (ii) a recombinant VAI gene, alone or in further combination with a recombinant VAII gene, wherein the recombinant gene comprises either of a regulatable promoter in place of the gene's native promoter or a mutated native promoter and, 5′ to the mutated native promoter, a pol II promoter, and, optionally, (iii) a pol II construct comprising a pol II promoter operably linked to a coding region and/or a pol III construct comprising a pol III promoter operably linked to a coding region, expression of VAI and/or VAII by the cell line may not be required. When the adenoviral vector comprises an adenoviral genome comprising (i) at least one deletion in a region of the adenoviral genome selected from the group consisting of E1, E2A and E4, (ii) an oligonucleotide sequence encoding a dominant negative, double-stranded PKR kinase, and optionally, (iii) a pol II construct comprising a pol II promoter operably linked to a coding region and/or a pol III construct comprising a pol III promoter operably linked to a coding region, the cell line need not express VAI, VAII or PKR kinase. When the adenoviral vector comprises (i) an adenoviral genome comprising at least one deletion in a region of the adenoviral genome selected from the group consisting of E1, E2A and E4, and, optionally, a pol II construct comprising a pol II promoter operably linked to a coding region and/or a pol III construct comprising a pol III promoter operably linked to a coding region, the cell line expresses a dominant negative, double-stranded PKR. The expression of the dominant negative, double-stranded PKR kinase can be constitutive, inducible or repressible as desired.

If a cell line is required for the above-described vector, such a cell line can be generated in accordance with standard molecular biological techniques. Those cell lines that contain the genes that complement for the deletions in the adenoviral vector are such that there is no overlap with respect to at least one essential gene function, which eliminates the possibility of the adenoviral vector genome recombining with the cellular DNA. Accordingly, replication-competent adenoviruses are eliminated from the vector stocks produced by such cell lines, which vector stocks are, therefore, suitable for certain therapeutic purposes, such as gene therapy or vaccines. This also prevents the replication of the adenoviruses in noncomplementing cells.

When the complementing cell line expresses the products of the at least one deletion in the E1, E2A and/or E4 regions and the at least one deletion in the VAI and/or VAII genes of the adenoviral genome, the complementing cell line must do so at the appropriate level for those products in order to generate a stock, preferably a high titer stock, of recombinant adenoviral vector. For example, it is necessary to express the E2A product, DBP, at stoichiometric levels, i.e., relatively high levels, for adenoviral DNA replication. Not only must the level of the product be appropriate, the temporal expression of the product must be consistent with that seen in normal viral infection of a cell in order to assure a stock, preferably a high titer stock, of recombinant adenoviral vector. For example, the components necessary for viral DNA replication must be expressed before those necessary for virion assembly. In order to avoid cellular toxicity, which often accompanies high levels of expression of the viral products, and to regulate the temporal expression of the products, regulatable promoter systems desirably are used. For example, the sheep metallothionine regulatable promoter system can be used to express the complete E4 region, the open reading frame 6 of the E4 region (see, e.g., U.S. Pat. No. 5,994,106), and the E2A region. Other examples of suitable regulatable promoter systems include, but are not limited to, the bacterial lac operon, the tetracycline operon, the T7 polymerase system, and combinations and chimeric constructs of eukaryotic and prokaryotic transcription factors, repressors and other components. Where the viral product to be expressed is highly toxic, it is desirable to use a bipartite inducible system, wherein the repressor is carried within the chromatin of the complementing cell line and the viral vector encodes the toxic gene product under the control of a repressible promoter. Repressible/inducible expression systems, such as the tetracycline expression system and lac expression system also can be used. When the promoter of the adenoviral VAI gene is regulatable, preferably it comprises an element that binds to a tetracycline repressor (see, e.g., U.S. Pat. No. 5,972,650). Preferably, the tetracycline repressor bound to the element interferes with the split binding sites of the transcription factor TFIIID. In some cell lines, it can be possible to use the VAI/VAII native promoter or a constitutive promoter to express the VAI/VAII gene product at the appropriate level without induction or repression.

DNA that enters a small proportion of transfected cells can become stably maintained in an even smaller fraction. Isolation of a cell line that expresses one or more transfected genes is achieved by introduction into the same cell of a second gene (marker gene) that, for example, confers resistance to an antibiotic, drug or other compound. This selection is based on the fact that, in the presence of the antibiotic, drug, or other compound, the cell without the transferred gene will die, while the cell containing the transferred gene will survive. The surviving cells are then clonally isolated and expanded as individual cell lines. Within these cell lines are those that will express both the marker gene and the genes of interest. Propagation of the cells is dependent on the parental cell line and the method of selection. Transfection of the cell is also dependent on cell type. The most common techniques used for transfection are calcium phosphate precipitation, liposome, or DEAE dextran mediated DNA transfer.

Thus, in view of the above, the invention further provides a method of making a cell line that complements an above-described adenoviral vector. The method comprises (i) introducing into the cell those regions of an adenoviral genome selected from the group consisting of E1, E2A and E4 that have been disrupted in the adenoviral vector, and (ii) introducing into the cell an adenoviral VAI coding region operably linked to a promoter and/or an adenoviral VAII coding region operably linked to a promoter, whereupon the cell expresses those regions of the adenoviral genome and the adenoviral VAI coding region and/or the adenoviral VAII coding region, as have been introduced into the cell, and desirably wherein there is no overlap with respect to at least one essential gene function between the adenoviral genome of the adenoviral vector and those regions of the adenoviral genome and the adenoviral VAI coding region and/or the adenoviral VAII coding region, as have been introduced into the cell for generation of replication-competent adenovirus, with the proviso that (i) and (ii) can be carried out in either order. If there is overlap with respect to the at least one essential gene function of the adenoviral genome of the adenoviral vector and those regions of the adenoviral genome and the adenoviral VAI and/or VAII coding region(s) as have been introduced into the cell, desirably the overlap is kept to a minimum. Preferably, the promoter is a native promoter or a regulatable promoter, such as one that comprises an element that binds to a tetracycline repressor. Preferably, the tetracycline repressor bound to the element interferes with the split binding sites of the transcription factor TFIIID. Alternatively and also preferably, the promoter is a VA promoter and the element that binds to a tetracycline repressor is incorporated 5′ to Box A of the VA promoter or between Box A and Box B of the VA promoter. In addition to the foregoing, a VA promoter which differs at the nucleotide level but maintains the same secondary and tertiary structure can be used.

A method of expressing a pol II construct comprising a pol II promoter operably linked to a coding region and/or a pol III construct comprising a pol III promoter operably linked to a coding region in a mammalian cell is also provided. The method comprises contacting the mammalian cell with an above-described adenoviral vector, which comprises a pol II construct comprising a pol II promoter operably linked to a coding region and/or a pol III construct comprising a pol III promoter operably linked to a coding region, whereupon the mammalian cell internalizes the adenoviral vector and expresses the pol II construct and/or the pol III construct in the mammalian cell. Preferably, the mammalian cell is in vivo.

A mammalian cell can be contacted in accordance with the above methods by any suitable means as known in the art. When the mammalian cell is in vivo, one skilled in the art will appreciate that suitable methods of administering an above-described adenoviral vector to a mammal for therapeutic purposes, e.g., gene therapy, vaccination, and the like (see, for example, Rosenfeld et al., Science 252: 431-434 (1991); Jaffee et al., Clin. Res. 39(2): 302A (1991); Rosenfeld et al., Clin. Res. 39(2): 311A (1991); and Berkner, BioTechniques 6: 616-629 (1988)), are available, and, although more than one route can be used to administer the vector, a particular route can provide a more immediate and more effective reaction than another route. A suitable carrier, such as a physiologically acceptable or pharmaceutically acceptable carrier, can be used to administer the vector, and suitable carriers are readily available and well-known to those who are skilled in the art. The choice of carrier will be determined in part by the particular method used to administer the vector. Accordingly, there is a wide variety of suitable formulations comprising the vector and a carrier therefor. The following formulations and methods of administration are merely exemplary and are in no way limiting.

Formulations suitable for oral administration can consist of (a) liquid solutions, such as an effective amount of the compound dissolved in diluents, such as water, saline, or orange juice; (b) capsules, sachets or tablets, each containing a predetermined amount of the active ingredient, as solids or granules; (c) suspensions in an appropriate liquid; and (d) suitable emulsions. The vectors of the invention, alone or in combination with other suitable components, can be made into aerosol formulations to be administered via inhalation. Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. See, e.g., U.S. Pat. No. 6,225,289 and WO 00/34444.

The amount of vector administered to a mammal, particularly a human, in the context of the invention will vary with the gene or other sequence of interest, the composition employed, the method of administration, and the particular site and organism being treated. The amount of vector administered should be sufficient to effect a desirable response, e.g., therapeutic or immune response, within a desirable time frame.

The adenoviral vectors and systems of the invention also have utility in vitro. For example, they can be used for interfering genomic studies (which also can be conducted in vivo).

### EXAMPLES

The following examples further illustrate the invention but, of course, should not be construed as in any way limiting its scope.

### Example 1

This example describes the construction of adenoviral vectors in which VAI is disrupted and VAII is partially or completely disrupted.

HEK 293 cells are obtained from the American Type Culture Collection (ATCC; CRL.1537) and are maintained in Dulbecco's modified Eagle's medium (DMEM) supplemented with 10% calf serum. HEK 293/ORF6 cells were constructed according to U.S. Pat. No. 5,851,806.

The 293VAI cell line and the 293/ORF6 VAI cell line are created by excising the 200 bp Xba I/Nhe I VAI Ad2 promoter and gene from pAdVantage, inserting the excised fragment into the eukaryotic expression vector pREP10 (Introgene), transfecting the resulting plasmid into 293 cells and 293/ORF6 cells, and selecting for hygromycin resistance and VA gene expression.

The 293VAI/VAII cell line is created by excising the 465 bp Xba I/Nsi I fragment containing the VAI/VAII Ad2 genes from pAdVantage, inserting the fragment into the eukaryotic expression vector pREP10, which is previously cut with Xba I and Sal I to eliminate the RSV promoter and the SV40 polyA sequences, transfecting the resulting plasmid (which retains the Nsi I and Sal I sites intact) into 293 cells and 293/ORF6 cells, and selecting for hygromycin resistance and VA gene expression.

The 293VAI and 293VAI/VAII cell lines are used to construct the VA-deleted vectors. The 293 VAI cell line has no overlapping sequences in the VAI region with the VA-deleted vectors, whereas the 293VAI/VAII cell line or the 293/ORF6 cell line has no overlapping sequence in the E4 region. Therefore, recombination between the cell line and the vector sequences is minimized, greatly reducing the possibility of replication-competent adenovirus (see Kovesdi et al., U.S. Pat. No. 5,994,106).

The control adenoviral vector, AdL, has been previously described (Brough et al., J. Virol. 70: 6497-6501 (1996); Brough et al., J. Virol. 71: 9206-9213 (1997)). The transgene expression is under the control of the cytomegalovirus immediate early promoter and luciferase is expressed (GenVec, Inc., Rockville, Md.).

The AdL(VA-) adenoviral vector expressing the luciferase gene is constructed by using the plasmid system described by McVey et al. (International Patent Application Publication No. WO 99/15686). In order to delete the VAI region and part of the VAII region in the vector, a shuttle vector plasmid (pDSC-VA) is constructed. pDSC-VA comprises a dual-selection cassette surrounded by homologous regions derived from sequences surrounding the VA region.

The pAdVantage vector is cut with Xba I and Fse I. The fragment comprising the Ad2 VAI gene and half of the VAII gene is replaced with a dual-selection cassette, thereby creating the pAdVantage (VA-) shuttle vector. The E1-, E3- and VA-deleted adenoviral vectors are generated using the method similar to McVey et al. (International Patent Application Publication No. WO 99/15686), in which the shuttle vector plasmid, pAdVantage (VA-), is transfected into E. coli with the pACE (E1.L) plasmid for homologous recombination, thereby generating the pACE(VA-) plasmid. This plasmid is purified, transfected into 293VA cells, propagated (Graham et al., J. Gen. Virol. 36: 59-77 (1977)), and purified through three sequential bandings on cesium chloride gradients. The purified virus is dialyzed against a buffer and stored at −80° C. until use (see, e.g., U.S. Pat. No. 6,225,289). Less than 1 in 1×10⁷ plaque-forming units of replication-competent adenovirus is expected to be present.

For vectors of this example, the cell line expresses the VA RNA transcript. The cell line can express VAI under the control of the native promoter or under the control of a regulatable promoter. For example, the tet-repressible system can be placed in the A or B box of the VAI promoter and VA production is repressed when the cell is growing and induced, by derepressing tet, when production of virus is desired.

### Example 2

This example describes the construction of adenoviral vectors in which precise deletions are made in each of the VAI and VAII genes.

Similarly to Example 1, vectors have been created by using Ad5, instead of Ad2, sequences. In this case, an Ad5 VA shuttle plasmid was used instead of pAdVantage (pVA1+2+). The pVA1+2+ plasmid contains the Ad5 sequences from Sac I (nt 10,267) to Hind III (nt 11,565). Specific regions of VAI and/or VAII were precisely deleted by standard PCR methodology. An adenovirus was constructed with previously known VAI and VAII partial deletions, which individually do not completely inactivate VA functions were combined into a single construct which has no VAI or VAII activities. The VAI promoter deletion from the mutant virus dl331 (29 bp deletion within VAI sequence, at +49 to +79 relative to transcription start site) was combined with the VAII promoter deletion of dl328 (17 bp deletion within VAII sequence, at +56 to +74 relative to transcription start site).

This adenovirus has overlapping sequences in the VAI region with the VAI complementing cell line.

### Example 3

This example describes the construction of adenoviral vectors in which VAI alone is under the control of a regulatable pol II promoter or in which both of VAI and VAII are under the control of a regulatable promoter.

Mutated oligonucleotides that comprise the VAI sequences from Ad5 were annealed and cloned into the pKSII plasmid vector (Stratagene) that was cut with Bam HI/Pst I creating plasmid pKSVAIM1. Four consecutive point mutations were incorporated into the oligonucleotides to inactivate the VAI promoter. The promoter mutations are at location 10632 to 10635 in the Ad5 sequence and resulted in a change from GTGG to CGCA. The complementing mutations to maintain the stem loop structure are at location 10759 to 10762 in the Ad5 sequence and changed the sequence CAAC to TGCG.

The mutated VAI sequence was excised from pKSVAIM1 and cloned between the promoter from the sheep metallothionein protein 1a gene (sMT-Ia gene), which is inducible with zinc, and the Herpes simplex TK poly-adenylation signal. The Ad5 sequences are from 10620 to 10775 inclusive. Ad5 bp 10620 is proximal to the promoter to generate pSMT-VAIM1.

The portion of the plasmid comprising the sMT-VA-TK sequences were reinserted into the pACE(VA-) plasmid of Example 1 and adenovirus was produced in 293 cells which were grown in media supplemented with 100 μM ZnCl₂.

The VAII gene can be placed under the control of a regulatable pol II promoter alone or in combination with the VAI construct described above.

The vectors of this example do not require a VAI or VAII complementing cell line.

### Example 4

This example describes the construction of adenoviral vectors with disruptions in the E1 and E3 regions of the adenoviral genome and with disruptions in the VAI and VAII genes.

Adenovirus 5 (Ad5) based vector genomes were constructed using the methods of McVey et al., J. Virol. 76(8): 3670-3677 (2002), U.S. Pat. No. 6,329,200, and international patent application WO 99/15686. The recipient plasmid pACE1(L)E3(10X)VA(ZZeo) is comprised of the Ad5 genome cloned into pACYC177 (New England BioLabs) between the Drd I and Dra I sites to retain the p15 origin of replication and the kanamycin resistance gene. The adenoviral left ITR is nearer to the Dra I site. The lambda phage cos packaging site is 32 bp from the adenoviral left ITR. The Lac I^{Q} expression cassette is oriented to direct transcription toward the cos site. Pac I restriction sites reside next to the 5′ end of the Lac I^{Q} gene and right adenoviral ITR. The luciferase gene under the control of the CMV promoter and SV40 polyadenylation signal replaces E1 sequences 356-3,327. The adenoviral genome also contains a deletion in the E3 region spanning base pairs 28,597 to 30,470. Adenoviral sequences 10,546-11,050 were replaced with a Pst I/Sal I fragment-containing EM7 Sc-ble portion of pCMV/Zeo (Invitrogen, Inc.). This fragment had been modified to comprise DNA sequences that encode the amino-terminal 57 amino acids of LacZ cloned immediately 3′ of the EM-7 promoter inframe with Sc-ble. The viral genome is also deleted for bp 10,594 and 10,595.

Vector genomes were constructed with pACE1(L)E3(10X)VA(ZZeo) and a series of VA shuttle plasmids that contain various deletions. The VA regions of Ad5 sequences 10,293-11,769 were amplified by PCR. The PCR product was restricted with Sac I and Hind III and cloned into the same sites in pBlueskriptKSH+ to generate pKSVA1+2+. From pKSVA1+2+ a series of shuttle VA deletion plasmids were constructed using a series of overlapping oligonucleotides using standard molecular biology techniques. The shuttle plasmids and their deletions are as follows: for pKSVA1−2+, Ad5 base pairs 10,620-10,779 inclusive were deleted, for pKSVA1−2−, Ad5 base pairs 10,620-10,779 and 10,928-10,944 inclusive were deleted, for pKSVA1b−2+, Ad5 base pairs 10,620-10,698 inclusive were deleted, and for pKSVA1b−2−, Ad5 base pairs 10,620-10,698 and 10,928-10,944 inclusive were deleted. The VA deletion shuttle plasmids were used to replace the Pst I/Sal I fragment-containing EM7 Sc-ble portion of pACE1(L)E3(10X)VA(ZZeo) by homologous recombination in E. coli.

pACE1(L)E3(10X)VA(1−2+) is isogenic to pACE1(L)E3(10X)VA(ZZeo) except in the VA region. The VA region is wild-type, except for the deletion of Ad5 base pairs 10,620-10,779 inclusive.

pACE1(L)E3(10X)VA(1b−2−) is isogenic to pACE1(L)E3(10X)VA(ZZeo) except in the VA region. The VA region is wild-type, except for the deletion of Ad5 base pairs 10,620-10,698 and 10,928-10,944 inclusive.

These pACE plasmids were used to generate E1- adenoviral vectors AdL.VA(1−2+) and AdL.VA(1b−2+) in 293 cells using the method of McVey et al. (2002), supra. The AdL.VA(1−2+) vector was purified on three CsCl gradients and dialyzed against storage buffer. The vector grew as other viral vectors, without the need for a VA trans-complementing cell line.

pACE1(L)E3(10)VA(1−2+)E4(BGGus) is isogenic to pACE1(L)E3(10X)VA(1−2+) except that the E3 deletion is from Ad5 base pairs 28,593-30,470 and Ad5 E4 base pairs 32,832-35,564 were replaced with the β-glucuronidase gene flanked by the bovine growth hormone (BGH) and SV40 polyadenylation sequences at its 5′ terminus and 3′ terminus, respectively.

pACE1(L)E3(10X)VA(1b−2−)E4(BGGus) is isogenic to pACE1(L)E3(10X)VA(1b−2−) except that the E3 deletion is from Ad5 base pairs 28,593-30,470 and Ad5 E4 base pairs 32,832-35,564 were replaced with the β-glucuronidase gene flanked by the BGH and SV40 polyadenylation sequences at its 5′ terminus and 3′ terminus, respectively.

These pACE plasmids were used to generate the E1-E4- adenoviral vectors AdL.VA(1−2−) and AdL.VA(1b−2−) in 293-ORF6 cells using the method of McVey (2002), supra. The AdL.VA(1−2−) vector was purified on three CsCl gradients and dialyzed against storage buffer. The vector grew without the need for a VA trans-complementing cell line, albeit at a lower level of production.

### Example 5

This example describes the construction of adenoviral vectors with disruptions in the E1, E3 and E4 regions of the adenoviral genome and with disruptions in the VAI and VAII genes.

Similarly to Example 4, vector genomes were constructed using the method of McVey et al. (2002), supra. pACE1 (L)E3(10)VA(1−2+)E4(BGGus) is isogenic to pACE1(L)E3(10X)VA(1−2+), except that the E3 deletion is from Ad5 base pairs 28,598-30,475 and Ad5 E4 base pairs 32,832-35,564 were replaced with the β-glucuronidase gene flanked by the BGH and SV40 polyadenylation sequences at its 5′ terminus and 3′ terminus, respectively.

pACE1(L)E3(10X)VA(1b−2−)E4(BGGus) is isogenic to pACE1(L)E3(10)VA(1b−2−), except that the E3 deletion is from Ad5 base pairs 28,598-30,475 and Ad5 E4 base pairs 32,832-35,564 were replaced with the β-glucuronidase gene flanked by the BGH and SV40 polyadenylation sequences at its 5′ terminus and 3′ terminus, respectively.

These pACE plasmids were used to generate the E1⁻E4⁻ adenoviral vectors AdL.VA(1−2−) and AdL.VA(1b−2−) in 293-ORF6 cells, using the method of McVey (2002), supra. The AdL.VA(1−2−) vector was purified on three CsCl gradients and dialyzed against storage buffer. The vector grew without the need for a VA trans-complementing cell line, albeit at a lower level of production.

All of the references cited herein, including patents, patent applications, and publications, are hereby incorporated in their entireties by reference, including U.S. provisional patent application No. 60/318,997, to which this application claims priority.

While this invention has been described with an emphasis upon preferred embodiments, variations of the preferred embodiments may be used, and it is intended that the invention may be practiced otherwise than as specifically described herein. Accordingly, this invention includes all modifications encompassed within the spirit and scope of the invention as defined by the following claims.

What is claimed is:

1. An adenoviral vector comprising an adenoviral genome comprising (i) at least one deletion in a region of the adenoviral genome selected from the group consisting of E1, E2A and E4, (ii) at least one deletion in the VAI gene of the adenoviral genome, alone or in further combination with at least one deletion in the VAII gene of the adenoviral genome, and, optionally, (iii) a polymerase II (pol II) construct comprising a pol II promoter operably linked to a coding region and/or a polymerase III (pol III) construct comprising a pol III promoter operably linked to a coding region.

2. The adenoviral vector of claim 1, which comprises at least one deletion in each of two regions of the adenoviral genome selected from the group consisting of E1, E2A and E4.

3. The adenoviral vector of claim 1, which comprises at least one deletion in each of the E1, E4, VAI and VAII regions of the adenoviral genome.

4. The adenoviral vector of claim 1, wherein the coding region operably linked to the pol III promoter encodes an antisense RNA molecule, a ribozyme or an RNA interfering molecule.

5. The adenoviral vector of claim 1, wherein the pol III promoter is a VA RNA promoter, a transfer RNA promoter or a small nuclear RNA promoter.

6. The adenoviral vector of claim 1, wherein the adenoviral vector is packaged as a virion.

7. An adenoviral vector comprising an adenoviral genome comprising (i) at least one deletion in a region of the adenoviral genome selected from the group consisting of E1, E2A and E4, (ii) a recombinant VAI gene, alone or in further combination with a recombinant VAII gene, wherein the recombinant gene comprises either of a regulatable promoter in place of the native promoter or a mutated native promoter and 5′ to the mutated native promoter, a pol II promoter, and, optionally, (iii) a pol II construct comprising a pol II promoter operably linked to a coding region and/or a pol III construct comprising a pol III promoter operably linked to a coding region.

8. The adenoviral vector of claim 7, which comprises at least one deletion in each of two regions of the adenoviral genome selected from the group consisting of E1, E2A and E4.

9. The adenoviral vector of claim 7, wherein the coding region operably linked to the pol III promoter encodes an antisense RNA molecule, a ribozyme, a small interfering RNA (siRNA), or any other RNA interfering molecule.

10. The adenoviral vector of claim 7, wherein the pol III promoter is a VA RNA promoter, a transfer RNA promoter or a small nuclear RNA promoter.

11. The adenoviral vector of claim 7, wherein the adenoviral vector is packaged as a virion.

12. An adenoviral vector comprising an adenoviral genome comprising (i) at least one deletion in a region of the adenoviral genome selected from the group consisting of E1, E2A and E4, (ii) a dominant negative, double-stranded, RNA-dependent protein kinase (PKR), and, optionally, (iii) a pol II construct comprising a pol II promoter operably linked to a coding region and/or a pol III construct comprising a pol III promoter operably linked to a coding region.

13. The adenoviral vector of claim 12, which comprises at least one deletion in each of two regions of the adenoviral genome selected from the group consisting of E1, E2A and E4.

14. The adenoviral vector of claim 12, wherein the coding region operably linked to the pol III promoter encodes an antisense RNA molecule, a ribozyme, an siRNA, or any other RNA interfering molecule.

15. The adenoviral vector of claim 12, wherein the pol III promoter is a VA RNA promoter, a transfer RNA promoter or a small nuclear RNA promoter.

16. The adenoviral vector of claims 12, wherein the adenoviral vector is packaged as a virion.

17. A system comprising an adenoviral vector of claim 1 and a cell line that complements the adenoviral vector, wherein the cell line comprises the adenoviral vector.

18. The system of claim 17, wherein the cell line that complements the adenoviral vector comprises and expresses an adenoviral VAI gene and/or VAII gene.

19. The system of claim 18, wherein the promoter of the adenoviral VAI gene is a native promoter.

20. The system of claim 18, wherein the promoter of the adenoviral VAI gene is a regulatable promoter.

21. The system of claim 20, wherein the promoter of the adenoviral VAI gene comprises an element that binds to a tetracycline repressor.

22. The system of claim 21, wherein the element that binds to a tetracycline repressor interferes with the split binding sites of the transcription factor TFIIID.

23. A system comprising an adenoviral vector of claim 7 and a cell line that complements the adenoviral vector, wherein the cell line comprises the adenoviral vector.

24. The system of claim 23, wherein the cell line that complements the adenoviral vector comprises and expresses an adenoviral VAI gene and/or VAII gene.

25. The system of claim 24, wherein the promoter of the adenoviral VAI gene is a native promoter.

26. The system of claim 24, wherein the promoter of the adenoviral VAI gene is a regulatable promoter.

27. The system of claim 26, wherein the promoter of the adenoviral VAI gene comprises an element that binds to a tetracycline repressor.

28. The system of claim 27, wherein the element that binds to a tetracycline repressor interferes with the split binding sites of the transcription factor TFIIID.

29. A system comprising the adenoviral vector of claim 12 and a cell line that complements the adenoviral vector, wherein the cell line comprises the adenoviral vector.

30. A system comprising an adenoviral vector comprising (i) an adenoviral genome comprising at least one deletion in a region of the adenoviral genome selected from the group consisting of E1, E2A and E4, and, optionally, a pol II construct comprising a pol II promoter operably linked to a coding region and/or a pol III construct comprising a pol III promoter operably linked to a coding region, and (ii) a cell line that complements the adenoviral vector, wherein the cell line expresses a dominant negative, double-stranded PKR and comprises the adenoviral vector.

31. The system of claim 30, in which the adenoviral vector comprises at least one deletion in each of two regions of the adenoviral genome selected from the group consisting of E1, E2A and E4.

32. The system of claim 30, in which the coding region operably linked to the pol III promoter encodes an antisense RNA molecule, a ribozyme, an siRNA, or any other RNA interfering molecule.

33. The system of claim 30, in which the pol III promoter is a VA RNA promoter, a transfer RNA promoter or a small nuclear RNA promoter.

34. The system of claim 30, in which the adenoviral vector is packaged as a virion.

35. A method of making an adenoviral vector of claim 1, which method comprises (i) introducing at least one deletion in a region of the adenoviral genome selected from the group consisting of E1, E2A and E4, (ii) introducing at least one deletion in the VAI gene of the adenoviral genome, alone or in further combination with at least one deletion in the VAII gene of the adenoviral genome, and, optionally, (iii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order.

36. The method of claim 35, which method further comprises (iv) propagating the adenoviral vector in a complementing cell line.

37. A method of making an adenoviral vector of claim 3, which method comprises (i) introducing at least one deletion in each of the E1, E4, VAI and VAII regions of the adenoviral genome, and, optionally, (ii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order.

38. The method of claim 37, which method further comprises (iii) propagating the adenoviral vector in a complementing cell line.

39. A method of making an adenoviral vector of claim 7, which method comprises (i) introducing at least one deletion in a region of the adenoviral genome selected from the group consisting of E1, E2A and E4, (ii) either (a) introducing a mutation into the native promoter of the VAI gene and/or the VAII gene, and 5′ to the mutated native promoter, introducing a pol II promoter, or (b) substituting a regulatable promoter for the native promoter of the VAI gene and/or the VAII gene, and, optionally, (iii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order.

40. The method of claim 39, which method further comprises (iv) propagating the adenoviral vector in a complementing cell line, which does not express VAI and VAII, in the presence of an inducer of the regulatable promoter.

41. A method of making an adenoviral vector of claim 12, which method comprises (i) introducing at least one deletion in a region of the adenoviral genome selected from the group consisting of E1, E2A and E4, (ii) introducing an oligonucleotide sequence encoding a dominant negative, double-stranded PKR kinase into the adenoviral vector and, optionally, (iii) introducing a pol II construct comprising a pol II promoter operably linked to a coding region and/or a pol III construct comprising a pol III promoter operably linked to a coding region into the adenoviral vector, with the proviso that (i) and (ii) can be carried out in either order.

42. The method of claim 41, which method further comprises (iv) propagating the adenoviral vector in a complementing cell line.

43. A method of expressing a pol II construct comprising a pol II promoter operably linked to a coding region and/or a pol III construct comprising a pol III promoter operably linked to a coding region in a mammalian cell, which method comprises contacting the mammalian cell with an adenoviral vector of claim 1, which comprises a pol II construct comprising a pol II promoter operably linked to a coding region and/or a pol III construct comprising a pol III promoter operably linked to a coding region, whereupon the mammalian cell internalizes the adenoviral vector and expresses the pol II construct and/or the pol III construct in the mammalian cell.

44. The method of claim 43, wherein the mammalian cell is in vivo.

45. A method of expressing a pol II construct comprising a pol II promoter operably linked to a coding region and/or a pol III construct comprising a pol III promoter operably linked to a coding region in a mammalian cell, which method comprises contacting the mammalian cell with an adenoviral vector of claim 3, which comprises a pol II construct comprising a pol II promoter operably linked to a coding region and/or a pol III construct comprising a pol III promoter operably linked to a coding region, whereupon the mammalian cell internalizes the adenoviral vector and expresses the pol II construct and/or the pol III construct in the mammalian cell.

46. The method of claim 45, wherein the mammalian cell is in vivo.

47. A method of expressing a pol II construct comprising a pol II promoter operably linked to a coding region and/or a pol III construct comprising a pol III promoter operably linked to a coding region in a mammalian cell, which method comprises contacting the mammalian cell with an adenoviral vector of claim 7, which comprises a pol II construct comprising a pol II promoter operably linked to a coding region and/or a pol III construct comprising a pol III promoter operably linked to a coding region, whereupon the mammalian cell internalizes the adenoviral vector and expresses the pol II construct and/or the pol III construct in the mammalian cell.

48. The method of claim 47, wherein the mammalian cell is in vivo.

49. A method of expressing a pol II construct comprising a pol II promoter operably linked to a coding region and/or a pol III construct comprising a pol III promoter operably linked to a coding region in a mammalian cell, which method comprises contacting the mammalian cell with an adenoviral vector of claim 12, which comprises a pol II construct comprising a pol II promoter operably linked to a coding region and/or a pol III construct comprising a pol III promoter operably linked to a coding region, whereupon the mammalian cell internalizes the adenoviral vector and expresses the pol II construct and/or the pol III construct in the mammalian cell.

50. The method of claim 49, wherein the mammalian cell is in vivo.

51. A method of making a cell line that complements the adenoviral vector of claim 1, which method comprises (i) introducing into the cell those regions of an adenoviral genome selected from the group consisting of E1, E2A and E4 that have been disrupted in the adenoviral vector, and (ii) introducing into the cell an adenoviral VAI coding region operably linked to a promoter and/or an adenoviral VAII coding region operably linked to a promoter desirably wherein there is insufficient overlap between the adenoviral genome of the adenoviral vector and those regions of the adenoviral genome and the adenoviral VAI coding region and/or the adenoviral VAII coding region, as have been introduced into the cell, for generation of replication-competent adenovirus, with the proviso that (i) and (ii) can be carried out in either order.

52. The method of claim 51, wherein the promoter operably linked to the adenoviral VAI coding region and/or the adenoviral VAII coding region is a native promoter.

53. The method of claim 51, wherein the promoter operably linked to the adenoviral VAI coding region and/or the adenoviral VAII coding region is a regulatable promoter.

54. The method of claim 53, wherein the promoter comprises an element that binds to a tetracycline repressor.

55. The method of claim 54, wherein the tetracycline repressor, when bound to the element, interferes with the split binding sites of the transcription factor TFIIID.

56. The method of claim 54, wherein the promoter is a VA promoter and the element that binds to a tetracycline repressor is incorporated 5′ to Box A of the VA promoter.

57. The method of claim 54, wherein the promoter is a VA promoter and the element that binds to a tetracycline repressor is incorporated between Box A and Box B of the VA promoter.
